(19) United States
(12) Patent Application Publication
(10) Pub. No.: US 2005/0103357 A1
(43) Pub. Date: May 19, 2005

(54) Disposable sand cap for removing calluses and callus removal device having the same

(75) Inventors: Jeom-Sup Jo, Jackson Heights, NY (US); Chang-Sung Ahn, Seoul (KR)

Correspondence Address: John S. Egbert;Harrison & Egbert, 7th Floor, 412 Main Street, Houston, TX, 77002, US

(73) Assignee: Jeom-Sup JO, Jackson Heights, NY (US)

(21) Appl. No.: 10/970,450

(22) Filed: Oct. 21, 2004

(30) Foreign Application Priority Data
Nov. 13, 2003 (KR) 20-2003-35563

(51) Int. Cl. A45D 29/18; A61B 17/00

(52) U.S. Cl. 132/76.4; 606/190

(57) ABSTRACT

The present invention provides a disposable sand cap for removing calluses and a callus removal device having the same. The disposable sand cap for removing calluses includes a cap body which has a “ [symbol] ”-shaped cross-section and is made of a synthetic resin, and an abrasive sand which has a 60˜90 mesh particle size and is attached on a lower end surface of the cap body with a bonding agent. The callus removal device has the disposable sand cap with the abrasive sand. The callus removal device includes a grip part having an elliptical shape, with a plurality of finger grooves provided at several predetermined positions around a circumferential outer surface of the grip part, thus allowing fingers of a user to be placed on finger grooves. The callus removal device further includes a sand cap support part extending downwards from a lower portion of the grip part, with an insert ring provided around a circumferential outer surface of the sand cap support part. The callus removal device further includes an intermediate depression part provided between the grip part and the sand cap support part while being depressed inwards.

### RELATED U.S. APPLICATIONS

Not applicable.

### STATEMENT REGARDING FEDERALLY SPONSORED RESEARCH OR DEVELOPMENT

Not applicable.

### REFERENCE TO MICROFICHE APPENDIX

Not applicable.

### FIELD OF THE INVENTION

The present invention relates generally to disposable sand caps for removing calluses and callus removal devices having the same, and more particularly, to a disposable sand cap for removing calluses and a callus removal device having the same, which are made of synthetic resins, thus reducing the manufacturing costs, and in which an abrasive sand is placed on an outer surface of the callus removal device, thus efficiently removing calluses over a wide region, and in which, once the use of the callus removal device is completed, the disposable sand cap is simply separated from the callus removal device and thrown away, so that the callus removal device does not infect other people with bacilli, thus being sanitary and being convenient to users.

### BACKGROUND OF THE INVENTION

Generally, keratosis palmaris et plantaris is defined as any condition of the skin characterized by an overgrowth of calluses on the palms or the soles of the feet. The cause of the above-mentioned condition is different according to a shape and distribution of each callus, heredity, an attack time, accompanying abnormal symptoms, progression and prognosis.

The calluses are caused by keratinization of epidermal tissues of the skin and crack due to changing seasons. Thus, dermis underneath the calluses may be damaged. To avoid the above-mentioned problems, it is necessary to periodically remove the calluses prior to the condition becoming more serious.

To remove the calluses, callus removal devices were proposed and have been used. The conventional callus removal devices for removing the calluses include abrasive sands which are integrally coupled to rotating shafts of motors.

However, in the conventional callus removal devices, after a user uses the callus removal device, callus remnants remain on the abrasive sand. Even though the abrasive sand is disinfected, the callus remnants remaining on the abrasive sand can become rotten. When the user reuses the callus removal device, the rotten callus remnants remaining on the abrasive sand are in contact with the palms and soles of the user. Thus, the palms and soles of the user may be contaminated with bacilli. As such, the conventional callus removal devices are unsanitary.

Furthermore, the use of the conventional callus removal device in public service places may lead to the communication of condition. Thus, the United States of America, and several states of Europe prohibit the use of the conventional callus removal device in public service places.

### BRIEF SUMMARY OF THE INVENTION

Accordingly, the present invention has been made keeping in mind the above problems occurring in the prior art, and an object of the present invention is to provide a disposable sand cap for removing calluses and a callus removal device having the same, which are made of synthetic resins, thus reducing the manufacturing costs, and in which an abrasive sand is placed on an outer surface of the callus removal device, thus efficiently removing calluses over a wide region, and in which, once the use of the callus removal device is completed, the disposable sand cap is simply separated from the callus removal device and thrown away, so that the callus removal device does not infect other people with bacilli, thus being sanitary and being convenient to users.

In an aspect, the present invention provides a disposable sand cap for removing calluses, to be held on a callus removal device. The disposable sand cap includes a cap body having a “ [symbol] ”-shaped cross-section and made of a synthetic resin, with a coupling groove provided around an inner surface of a sidewall of the cap body; and an abrasive sand having a 60˜90 mesh particle size and attached to a lower surface of the cap body by a bonding agent.

In another aspect, the present invention provides a callus removal device having a disposable sand cap with an abrasive sand for removing calluses. The callus removal device includes a sand body. The sand body has a grip part having an elliptical shape, with a plurality of finger grooves provided at several predetermined positions around a circumferential outer surface of the grip part, thus allowing fingers of a user to be placed in finger grooves; a sand cap support part extending downwards from a lower portion of the grip part, with an insert ring provided around a circumferential outer surface of the sand cap support part; and an intermediate depression part provided between the grip part and the sand cap support part while being depressed inwards.

The sand body may be vertically divided into two parts, and the sand body may further include a coupling protrusion and a coupling boss protruded in the two parts of the sand body at positions corresponding to each other, thus engaging with each other.

### BRIEF DESCRIPTION OF THE SEVERAL VIEWS OF THE DRAWINGS

The above and other objects, features and other advantages of the present invention will be more clearly understood from the following detailed description taken in conjunction with the accompanying drawings, in which:

FIG. 1 is a bottom perspective view of a callus removal device having a disposable sand cap for removing calluses, according an embodiment of the present invention;

FIG. 2 is an exploded bottom perspective view of the callus removal device of FIG. 1; and

FIG. 3 is a sectional view of the callus removal device of FIG. 1.

### DETAILED DESCRIPTION OF THE INVENTION

Hereinafter, embodiments of the present invention will be described in detail with reference to the attached drawings.

Reference now should be made to the drawings, in which the same reference numerals are used throughout the different drawings to designate the same or similar components.

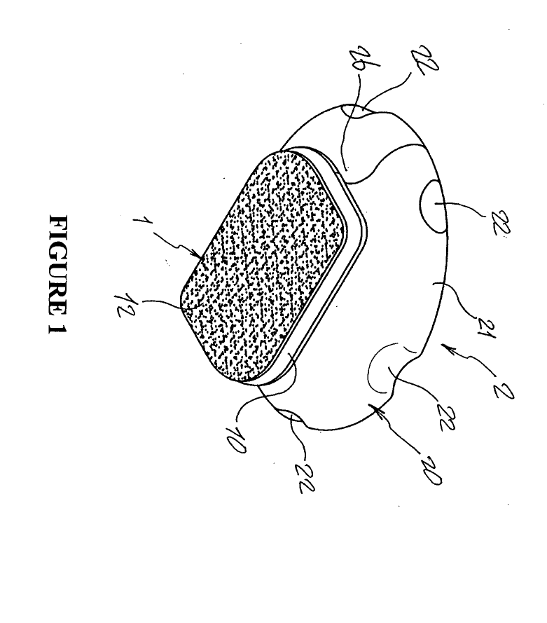

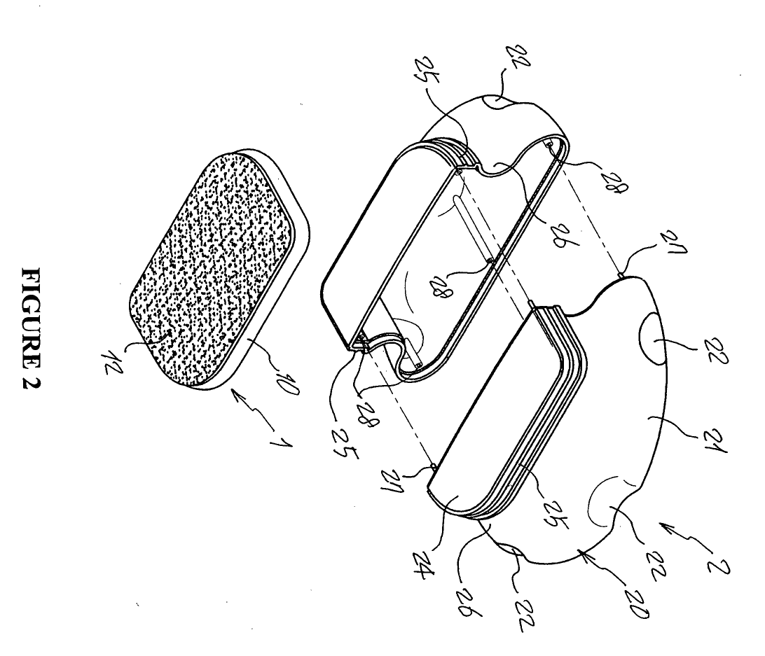

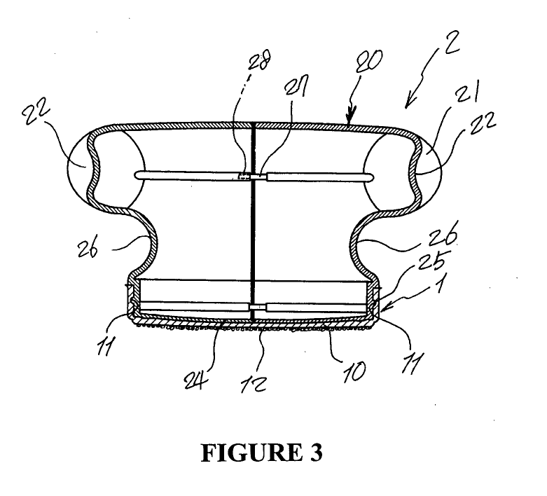

FIG. 1 is a bottom perspective view of a callus removal device 2 having a disposable sand cap 1 for removing calluses, according an embodiment of the present invention. FIG. 2 is an exploded bottom perspective view of the callus removal device 2 of FIG. 1. FIG. 3 is a sectional view of the callus removal device 2 of FIG. 1.

As shown in FIGS. 1 through 3, the disposable sand cap 1 for removing calluses according to the present invention includes a cap body 10 which has a “ [symbol] ”-shaped cross-section and is made of a synthetic resin, with a coupling groove 11 provided around an inner surface of a sidewall of the cap body 10. The disposable sand cap 1 for removing calluses further includes an abrasive sand 12 which has a 60˜90 mesh particle size and is attached to a lower surface of the cap body 10 by a bonding agent.

The callus removal device 2, on which the disposable sand cap 1 is held, includes a sand body 20. The sand body 20 includes a grip part 21 having an elliptical shape. A plurality of finger grooves 22 is provided at several predetermined positions around a circumferential outer surface of the grip part 21, thus allowing fingers of a user to be placed in finger grooves 22. The sand body 20 further includes a sand cap support part 24 which extends downwards from a lower portion of the grip part 21, with an insert ring 25 provided around a circumferential outer surface of the sand cap support part 24. The sand body 20 further includes an intermediate depression part 26 which is provided between the grip part 21 and the sand cap support part 24 while being depressed inwards.

The sand body 20 is vertically divided into two parts. The sand body 20 further includes a plurality of coupling protrusions 27 and a plurality of coupling bosses 28 which protrude from the two parts of the sand body 20 at positions corresponding to each other and engage with each other.

The sand cap 1 is disposable and is made of a hard synthetic resin, such as a hard PVC (polyvinyl chloride), etc. Preferably, the abrasive sand 12 having a 60˜100 mesh particle size is attached to the lower surface of the cap body 10 by the bonding agent to execute fine abrasion.

The bonding agent to attach the abrasive sand 12 to the cap body 10 preferably comprises a cold bonding agent.

The operation and effect of the callus removal device 2 having the disposable sand cap 1 according to the present invention will be described herein below.

To form the disposable sand cap 1, the cap body 10 having the “ [symbol] ”-shaped cross-section is formed with the hard synthetic resin, such as a hard PVC, such that the coupling groove 11 is provided around the inner surface of the sidewall of the cap body 10. Thereafter, the abrasive sand 12, such as an emery having the 60˜100 mesh particle size, is attached to the lower surface of the cap body 10 by a bonding agent to complete the forming of the disposable sand cap 1.

The callus removal device 2 comprises the disposable sand cap 1 and the sand body 20 divided into two parts, as shown in FIG. 2. The two parts are coupled to each other to provide the sand body 20 of a single body while the plurality of coupling protrusions 27 and coupling bosses 28, which are provided in the sand body 20 at positions corresponding to each other, engage with each other.

The sand body 20 has at an upper portion thereof the grip part 21. The finger grooves 22 are provided around the circumferential outer surface of the grip part 21, so as to allow the fingers of the user to be placed on the finger grooves 22 during the use of the callus removal device 2. The intermediate depression part 26 is provided under the grip part 21. Thus, when the user grasps the grip part 21, fingertips are placed on the intermediate depression part 26. Therefore, the user can firmly hold the callus removal device 2 during the use of the callus removal device 2.

The insert ring 25 is provided around the circumferential outer surface of the sand cap support part 24 which extends downwards from the lower portion of the grip part 21. As shown in FIG. 3, the insert ring 25 is inserted into the coupling groove 11 of the cap body 10 while the cap body 10 is forcibly fitted over the sand cap support part 24.

To use the callus removal device 2, the user grasps the grip part 21 of the sand body 20 and scrubs calluses off palms or soles with the abrasive sand 12 of the cap body 10.

Once the use of the callus removal device 2 is completed, the cap body 10 of the disposable sand cap 1 is removed from the lower end of the sand body 10 by being pulled out downwards.

As described above, the present invention provides a disposable sand cap for removing calluses and a callus removal device having the same, which are made of synthetic resins, thus reducing the manufacturing costs. Furthermore, the callus removal device of the present invention has at an outer surface an abrasive sand, thus efficiently removing calluses over a wide region. In addition, once the use of the callus removal device is completed, the disposable sand cap is simply separated from the callus removal device and thrown away. Therefore, the callus removal device does not infect other people with bacilli, thus being sanitary. Moreover, the callus removal device having the disposable sand cap is convenient to users.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

1. A disposable sand cap for removing calluses, to be held on a callus removal device, said disposable sand cap comprising:
a cap body having a “ [symbol] ”-shaped cross-section and being comprised of a synthetic resin, with a coupling groove provided around an inner surface of a sidewall of said cap body; and
an abrasive sand having a 60˜90 mesh particle size and attached to a lower surface of the cap body by a bonding agent.

2. A callus removal device comprising:
a disposable sand cap with an abrasive sand;
a sand body attachable to said disposable sand cap;
a grip part having an elliptical shape, with a plurality of finger grooves provided at several predetermined positions around a circumferential outer surface of the grip part, allowing fingers of a user to be placed on said finger grooves;
a sand cap support part extending downwards from a lower portion of the grip part, with an insert ring provided around a circumferential outer surface of the sand cap support part; and
an intermediate depression part provided between the grip part and the sand cap support part while being depressed inwards.

3. The callus removal device according to claim 2, wherein said sand body is vertically divided into two parts, and wherein said sand body further comprises a coupling protrusion and a coupling boss protruding from the two parts of the sand body at positions corresponding to each other, engageable with each other.
